(19) United States
(12) Patent Application Publication
(10) Pub. No.: US 2025/0177779 A1
(43) Pub. Date: Jun. 5, 2025

(54) RADIOMIC TUMOR DIVERSITY FEATURES IN BOWEL CANCERS

(71) Applicant: Case Western Reserve University, Cleveland, OH (US)

(72) Inventors: Sneha Singh, Shaker Heights, OH (US); Satish E. Viswanath, Beachwood, OH (US); Thomas Desilvio, Lyndhurst, OH (US)

(21) Appl. No.: 18/961,553

(22) Filed: Nov. 27, 2024

Related U.S. Application Data
(60) Provisional application No. 63/606,130, filed on Dec. 5, 2023.

(51) Int. Cl. A61N 5/10; G06T 7/00; G16H 20/40; G16H 30/40; G16H 50/20

(52) U.S. Cl. CPC A61N 5/1039; G06T 7/0016; G16H 20/40; G16H 30/40; G16H 50/20; G06T 2207/20081; G06T 2207/30028; G06T 2207/30096

(57) ABSTRACT

In some embodiments, the present disclosure relates to a method. The method includes extracting a plurality of pre-treatment features from one or more first regions of interest (ROI) within pre-treatment imaging data. Prognostic pre-treatment features are identified from the plurality of pre-treatment features. The prognostic pre-treatment features are determinative of a treatment response. A plurality of post-treatment features are extracted from one or more second ROI within post-treatment imaging data. Prognostic post-treatment features are extracted from the plurality of post-treatment features. The prognostic post-treatment features are determinative of the treatment response. Prognostic tumor diversity features are determined from a common subset of the prognostic pre-treatment features and the prognostic post-treatment features. A machine learning stage is operated to generate a medical prediction of the treatment response for a bowel cancer patient using the prognostic tumor diversity features.

### REFERENCE TO RELATED APPLICATION

This application claims the benefit of U.S. Provisional Application No. 63/606,130, filed on Dec. 5, 2023, the contents of which are incorporated by reference in their entirety.

### FEDERAL FUNDING NOTICE

This invention was made with government support under CA248226 awarded by the National Institutes of Health and W81XWH-21-1-0345 awarded by the Department of Defense. The government has certain rights in the invention.

### BACKGROUND

In recent years, there has been significant recent interest in developing machine vision tools for interrogating medical images. Machine vision tools are computer systems that utilize artificial intelligence to analyze medical images. Such systems have the potential to improve health care for patients.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a machine vision system configured to utilize tumor diversity features to generate a medical prediction of a treatment response for a bowel cancer patient.

FIG. 2 illustrates a flow diagram showing an exemplary method of training a machine vision system and operating the trained machine vision system to utilize tumor diversity features to generate a medical prediction of a treatment response for an additional bowel cancer patient.

FIG. 3 illustrates example box-plots showing tumor diversity feature values for different treatment responses associated with tumor diversity features extracted from pre-treatment and post-treatment imaging data.

FIG. 4 illustrates some additional embodiments of a machine vision system configured to utilize tumor diversity features to generate a medical prediction for a bowel cancer patient.

FIG. 5 illustrates some additional embodiments of a machine vision system configured to utilize tumor diversity features to generate a medical prediction for a bowel cancer patient.

FIGS. 6A-6B illustrate some embodiments of flow charts showing tumor diversity features being extracted from pre-treatment images and post treatment images.

FIG. 7 illustrates some additional embodiments of a machine vision system configured to utilize tumor diversity features to generate a medical prediction of a treatment response for a bowel cancer patient.

FIG. 8 illustrates some embodiments of a flow diagram showing a method for using tumor diversity features to generate a medical prediction of a treatment response for a rectal cancer patient.

FIG. 9 illustrates some embodiments of a radiomic analysis pipeline within a disclosed machine vision system configured to utilize tumor diversity features to generate a medical prediction of a treatment response for a rectal cancer patient.

FIG. 10 illustrates a block diagram of some embodiments of a prognostic apparatus configured to utilize tumor diversity features to generate a medical prediction of a treatment response for a bowel cancer patient.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Rectal cancer is a disease in which cancer cells develop in the rectum, which is part of the large intestine (e.g., the last several inches of the large intestine). Rectal cancer is estimated to account for about 10.2% of worldwide cancer incidence as well as about 9.4% of cancer-related deaths. It is projected that 3.2 million new cases of rectal cancer will be diagnosed globally by 2040. Rectal cancer is curable, especially when detected early through screening methods like a colonoscopy. Treatment for rectal cancer often combines surgery with chemotherapy and/or radiation, which can be given before or after surgery.

For example, neoadjuvant chemoradiation therapy followed by total mesorectal excision is a routine treatment for the patients with locally advanced rectal cancer. Beyond simply down staging rectal tumors prior to surgery, neoadjuvant chemoradiation therapy has been shown to result in a complete pathologic response (e.g., no residual tumor found on pathologic examination of surgical specimens) in between approximately 15% and approximately 27% of patients. Personalizing the treatment paradigm in rectal cancers to account for this response rate requires early and accurate identification of tumor regression in order to appropriately plan for personalized management of rectal cancer patients. For instance, patients exhibiting complete or near-complete response could be candidates for non-operative management including organ preservation (shown to have non-inferior survival compared to surgery, while dramatically improving their quality of life), while patients with minimal response despite neoadjuvant therapy may require additional chemoradiation therapy after surgery to minimize the chances of local recurrence.

The present disclosure relates an apparatus and/or method that utilizes a plurality of tumor diversity features, which have been extracted from one or more radiological images (e.g., MRI images) of a bowel cancer patient, to generate a medical prediction of the patient's response to treatment (e.g., neoadjuvant chemoradiation therapy). In some embodiments, the method comprises extracting a first plurality of tumor diversity features from pre-treatment imaging data and extracting a second plurality of tumor diversity features from post-treatment imaging data. The first and second plurality of tumor diversity features are analyzed to identify a plurality of tumor diversity features that are prognostic of a treatment response and that show a response to a treatment (e.g., that have pre-treatment and post-treatment values that are larger than a threshold). A machine learning model is trained using the plurality of tumor diversity features to generate a medical prediction of a treatment response. The tumor diversity features describe global and/or local structural attributes of tumors on radiological images. Therefore, by identifying tumor diversity features that are prognostic a treatment response and that show a response to treatment, the present disclosure is able to train a machine learning model to characterize tumor-related evolution and make an accurate prediction of treatment response for a bowel cancer patient.

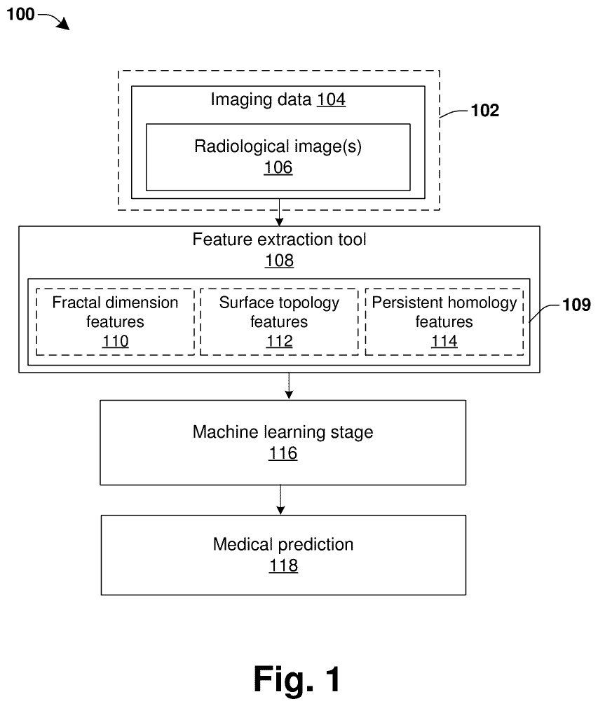

FIG. 1 illustrates some embodiments of a machine vision system 100 configured to utilize tumor diversity features to generate a medical prediction of a treatment response for a bowel cancer patient.

The machine vision system 100 comprises a memory 102 configured to store imaging data 104 for a bowel cancer patient (e.g., a rectal cancer patient, a colon cancer patient, a colorectal cancer patient, etc.). In some embodiments, the imaging data 104 may comprise one or more radiological images 106 (e.g., one or more MRI images, CT images, and/or the like). In various embodiments, the one or more radiological images 106 may comprise a pre-treatment image (e.g., an image of a patient taken prior to application of neoadjuvant chemoradiation therapy) and/or a post-treatment image (e.g., an image of a patient taken after application of neoadjuvant chemoradiation therapy).

A feature extraction tool 108 is configured to extract a plurality of tumor diversity features 109 from the imaging data 104. The plurality of tumor diversity features 109 include radiomic features that quantify surface and/or geometry differences driven by pathophysiological attributes of evolution of a treatment response (e.g., a chemoradiation response). For example, the plurality of tumor diversity features 109 may include radiomic features that describe changes in structural related patterns (e.g., complex and correlated patterns) within a tumor due to a treatment response. The changes may include uneven topological changes and surface differences due to irregular tumor growth or aggressive infiltration, which are hallmarks of treatment resistance. In some embodiments, the plurality of tumor diversity features 109 may include features that are both affected by the treatment (e.g., that have values that differ significantly before and after treatment) and are prognostic of a treatment response (e.g., that have values that differ significantly between a pathologic complete response and non-pathologic complete response).

In some embodiments, the plurality of tumor diversity features 109 may comprise fractal dimension features 110, surface topology features 112, and persistent homology features 114. The fractal dimension features 110 may comprise statistical measures (e.g., a standard deviation, a median, etc.) of fractal dimensions that are able to capture self-similar patterns within a tumor. The surface topology features 112 may represent one or more of an object sharpness, a shape index, a curvature, and a curvedness. The persistent homology features 114 may correspond to a number of active connected components with respect to varying dimensions and scales.

A machine learning stage 116 is configured to operate upon an input vector comprising the plurality of tumor diversity features 109 to generate a medical prediction 118 relating to a treatment response. In some embodiments, the medical prediction 118 may include a prediction that a bowel cancer patient will achieve a pathologic complete response or a non-pathologic complete response. Because the tumor diversity features 109 can accurately quantify geometry differences driven by pathophysiological attributes of evolution of a treatment response in cancers, the tumor diversity features 109 are able to generate the medical prediction 118 with a high degree of accuracy. In some embodiments, the medical prediction 118 may include an expected pathologic AJCC tumor regression grade in response to treatment, a ypTNM classification in response to treatment, and/or the like.

The medical prediction 118 may provide a health care professional with information that can be used to make a personalized treatment plan for a bowel cancer patient, which possibly avoids negative side effects and/or complications of treatment. For example, while neoadjuvant treatment in bowel cancer patients is often recommended as the standard of care, not all patients respond similarly to the treatment. Based upon the medical prediction 118, a doctor may decide that a patient that is likely to exhibit a pathologic complete response may receive non-operative management including organ preservation, while a patient that is likely to exhibit a non-pathologic complete response may require additional chemoradiation therapy after surgery to minimize the chances of local recurrence.

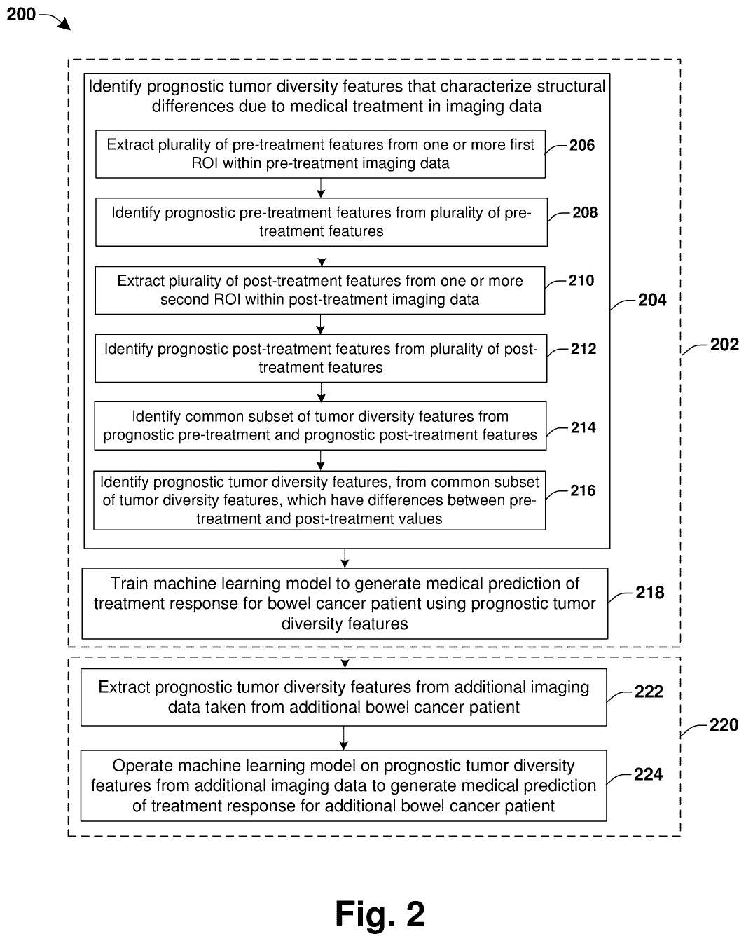

FIG. 2 illustrates a flow diagram showing an exemplary method 200 of training a machine vision system and operating the trained machine vision system to utilize tumor diversity features to generate a medical prediction of a treatment response for an additional bowel cancer patient.

While the disclosed methods (e.g., methods 200 and 800) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

The method 200 includes a training phase 202 and an application phase 220. In some embodiments, the training phase includes acts 204-218 and the application phase includes acts 222-224.

At act 204, a plurality of tumor diversity features, which characterize structural differences due to cancer treatment in imaging data, are identified. In some embodiments, the plurality of tumor diversity features are identified according to acts 206-216.

At act 206, a plurality of pre-treatment features are extracted from one or more regions of interest (ROI) within pre-treatment imaging data from a first plurality of bowel cancer patients.

At act 208, prognostic pre-treatment features are identified from the plurality of pre-treatment features. The prognostic pre-treatment features are features that are prognostic of a treatment response (e.g., that are prognostic of a pathologic complete response vs. a non-pathologic complete response) in the pre-treatment imaging data.

At act 210, a plurality of post-treatment features are extracted from one or more second ROI within post-treatment imaging data from a second plurality of bowel cancer patients.

At act 212, prognostic post-treatment features are identified from the plurality of post-treatment features. The prognostic post-treatment features are features that are prognostic of the treatment response in the post-treatment imaging data.

At act 214, a common subset of tumor diversity features are identified from the prognostic pre-treatment features and the prognostic post-treatment features. The common subset of tumor diversity features are features that are present in both the prognostic pre-treatment features and the prognostic post-treatment features.

At act 216, prognostic tumor diversity features, which have differences between pre-treatment and post-treatment values, are identified within the common subset of tumor diversity features. The differences between the pre-treatment and post-treatment feature values indicate that the prognostic tumor diversity features describe changes that are due to a treatment. In some embodiments, the prognostic tumor diversity features include features that have a difference in value that is over a threshold (e.g., 10%, 20%, 50%, or the like).

At act 218, a machine learning model is trained to generate a medical prediction of a treatment response for a bowel cancer patient using the plurality of tumor diversity features.

In some embodiments, the application phase 220 includes acts 222-224.

At act 222, the prognostic tumor diversity features are extracted from additional imaging data from an additional bowel cancer patient.

At act 224, a machine learning model is operated on the prognostic tumor diversity features extracted from the additional imaging data to generate a medical prediction of a treatment response for the additional bowel cancer patient.

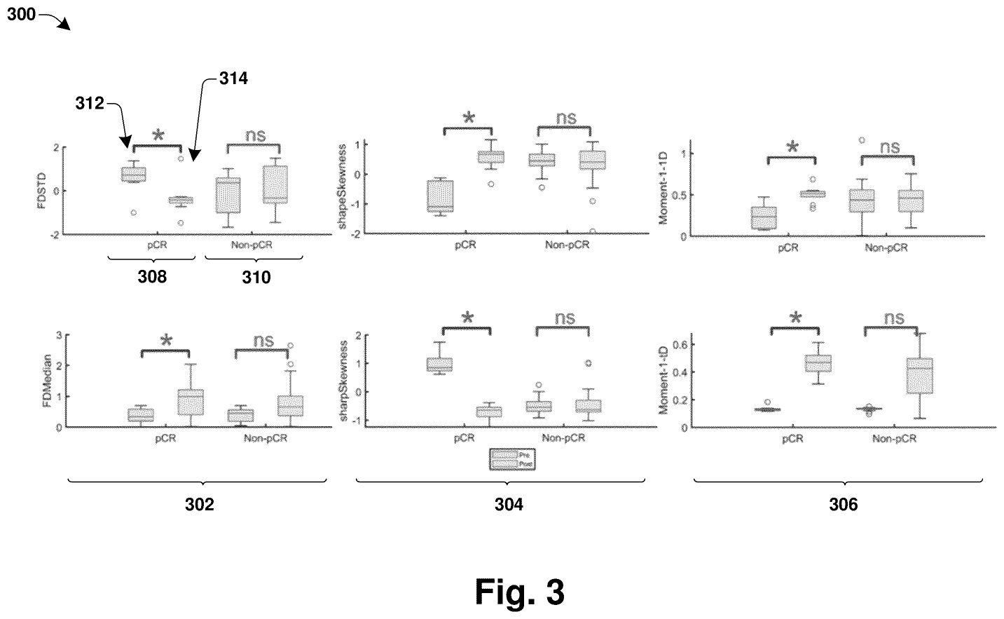

FIG. 3 illustrates example box-plots 300 showing tumor diversity feature values for different treatment responses associated with tumor diversity features extracted from pre-treatment and post-treatment imaging data.

The box-plots 300 correspond to tumor diversity features including fractal dimension features 302, surface topology features 304, and persistent homology features 306. For each of the box-plots 300, values of tumor diversity features are illustrated along a vertical axis (e.g., a y-axis) and a type of response is illustrated along a horizontal axis (e.g., an x-axis). The type of response is either a pathologic complete response (pcR) 308 or a non-pathologic complete response (non-pCR) 310. For each type of response, the box-plots 300 include a feature value range for a pre-treatment group 312 and a feature value range for a post treatment group 314. For example, each box plot illustrates a feature value range of a tumor diversity feature extracted from a pre-treatment image and a feature value range of a tumor diversity feature extracted from a post-treatment image for patients experiencing a pathologic complete response and for patients experiencing a non-pathologic complete response.

The box-plots 300 illustrate excellent separation between feature value ranges of groups of patients experiencing a pathologic complete response 308 and feature value ranges of groups of patients experiencing a non-pathologic complete response 310, thereby indicating that the features are prognostic of a type of response. The box-plots 300 also illustrate that feature value ranges of the tumor diversity features have a greater degree of change between pre-treatment and post-treatment images in groups of patients experiencing a pathologic complete response 308, than in groups of patients experiencing a non-pathologic complete response 310. The larger degree of change in the between feature value ranges in the pre-treatment groups 312 indicates that the features correspond to changes driven by the treatment.

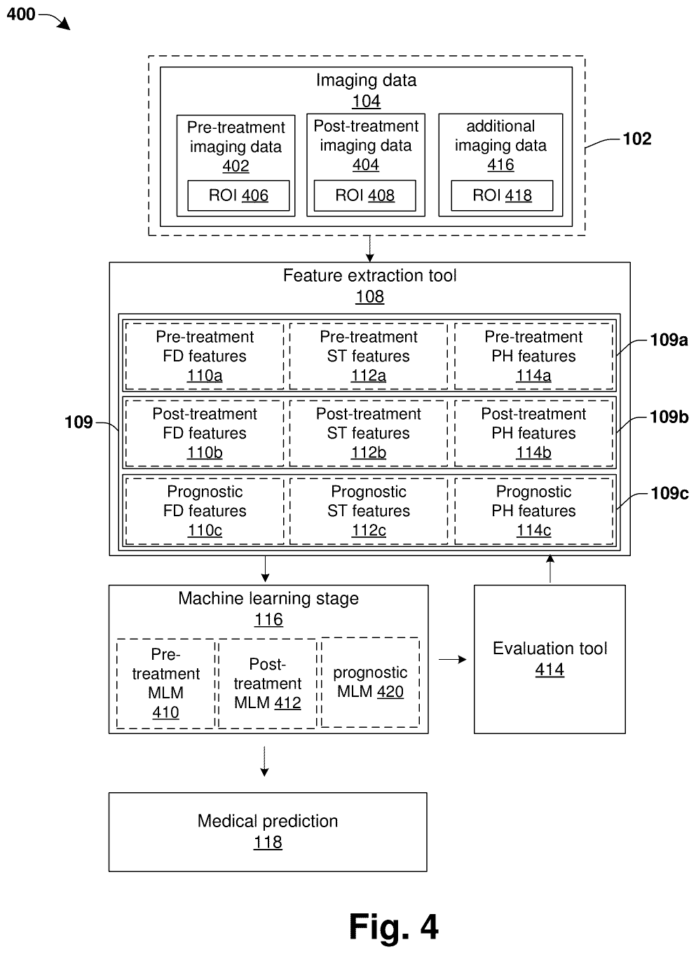

FIG. 4 illustrates some additional embodiments of a machine vision system 400 configured to utilize radiomic tumor diversity features to generate a medical prediction of a treatment response for a bowel cancer patient.

The machine vision system 400 comprises a memory 102 configured to store imaging data 104 from one or more bowel cancer patients. In some embodiments, the imaging data 104 may comprise pre-treatment imaging data 402 (e.g., taken prior to application of neoadjuvant chemoradiation therapy) and post-treatment imaging data 404 (e.g., taken after application of neoadjuvant chemoradiation therapy). The pre-treatment imaging data 402 and the post-treatment imaging data 404 may include images from a same patient and/or from different patients. In some embodiments, the memory 102 may comprise electronic memory (e.g., solid state memory, SRAM (static random-access memory), DRAM (dynamic random-access memory), and/or the like).

In some embodiments, the pre-treatment imaging data 402 and the post-treatment imaging data 404 may be segmented to identify one or more regions of interest (ROI). For example, the pre-treatment imaging data 402 may be segmented to identify one or more first ROI 406 and the post-treatment imaging data 404 may be segmented to identify one or more second ROI 408. In some embodiments, the one or more bowel cancer patients may be rectal cancer patients. In such embodiments, the imaging data 104 may respectively include a rectum of the rectal cancer patient, the one or more first ROI 406 may include a tumor region, and the one or more second ROI 408 may include a rectal wall.

In some additional embodiments, the imaging data 104 may further comprise additional imaging data 416 from an additional bowel cancer patient. The additional imaging data 416 may be segmented to identify one or more additional ROI 418. In some embodiments, the additional bowel cancer patient may be a rectal cancer patient and the one or more additional ROI 418 may include a tumor region.

A feature extraction tool 108 is configured to extract a plurality of tumor diversity features 109 from the imaging data 104. The plurality of tumor diversity features 109 may include a plurality of pre-treatment features 109a extracted from the one or more first ROI 406 within the pre-treatment imaging data 402 and a plurality of post-treatment features 109b extracted from the one or more second ROI 408 within the post-treatment imaging data 404. In some embodiments, the plurality of tumor diversity features 109 may include radiomic features that characterize structural features of the imaging data 104. In some embodiments, the plurality of tumor diversity features 109 may include radiomic features that describe changes in complex and correlated patterns due to a treatment response. In some embodiments, the feature extraction tool 108 may be run on one or more processors (e.g., a central processing unit including one or more transistor devices configured to operate computer code to achieve a result, a microcontroller, or the like).

In some embodiments, the plurality of tumor diversity features 109 may comprise fractal dimension features 110a-110c, surface topology features 112a-112c, and persistent homology features 114a-114c. In some embodiments, the fractal dimension features 110a-110c may comprise statistical measures (e.g., a standard deviation and a median) of fractal dimensions that are able to capture self-similar patterns within a tumor. In some embodiments, the surface topology features 112a-112c may represent one or more of an object sharpness, a shape index, a curvature, and a curvedness. In some embodiments, the persistent homology features 114a-114c correspond to a number of active connected components with respect to varying dimensions and scales (e.g., comprise Moment 1 of 1D and (0+1) D topological features)

A machine learning stage 116 is configured to operate upon an input vector comprising one or more of the plurality of tumor diversity features 109 to generate a medical prediction 118 corresponding to a treatment response. For example, the medical prediction 118 may be a prediction that a bowel cancer patient will experience a pathologic complete response (e.g., ypT0 or no evidence of tumor cells) or a non-pathologic complete response (e.g., ypT2 or a tumor having dimensions of between approximately 2 cm and approximately 5 cm) in response to treatment. In some embodiments, the machine learning stage 116 may be run on one or more processors (e.g., a central processing unit including one or more transistor devices configured to operate computer code to achieve a result, a microcontroller, or the like).

In some embodiments, the machine learning stage 116 may comprise a pre-treatment machine learning model 410 and a post-treatment machine learning model 412. The pre-treatment machine learning model 410 has been trained to operate on the pre-treatment features 109a to generate the medical prediction 118. The post-treatment machine learning model 412 has been trained to operate on the post-treatment features 109b to generate the medical prediction 118.

In some embodiments, the pre-treatment machine learning model 410 and the post-treatment machine learning model 412 may be configured to identify prognostic features that correspond to a treatment response. An evaluation tool 414 may be configured to perform statistical analysis of the prognostic features to determine prognostic tumor diversity features 109c. In some embodiments, the prognostic tumor diversity features 109c are features that are both prognostic of a treatment response (e.g., that have values that different significant between different treatment responses) and that correspond to a treatment (e.g., that have values that significantly different between the pre-treatment features 109a and the post-treatment features 109b). In some embodiments, the prognostic tumor diversity features may comprise a top 9 response-associated tumor diversity features.

The prognostic tumor diversity features 109c may be subsequently used to train a prognostic machine learning model 420 to generate the medical prediction 118. Once trained, the prognostic machine learning model 420 can be applied to tumor diversity features 109 (e.g., including the prognostic tumor diversity features 109c) extracted from one or more additional ROI 418 within additional imaging data 416 from an additional bowel cancer patient to generate the medical prediction 118 for the additional cancer patient. In some embodiments, the evaluation tool 414 may be configured to provide feedback to the feature extraction tool 108 concerning the prognostic tumor diversity features 109c, so that the feature extraction tool 108 can extract the prognostic tumor diversity features 109c from the additional imaging data 416 during operation of the machine vision system 400 on the additional bowel cancer patient.

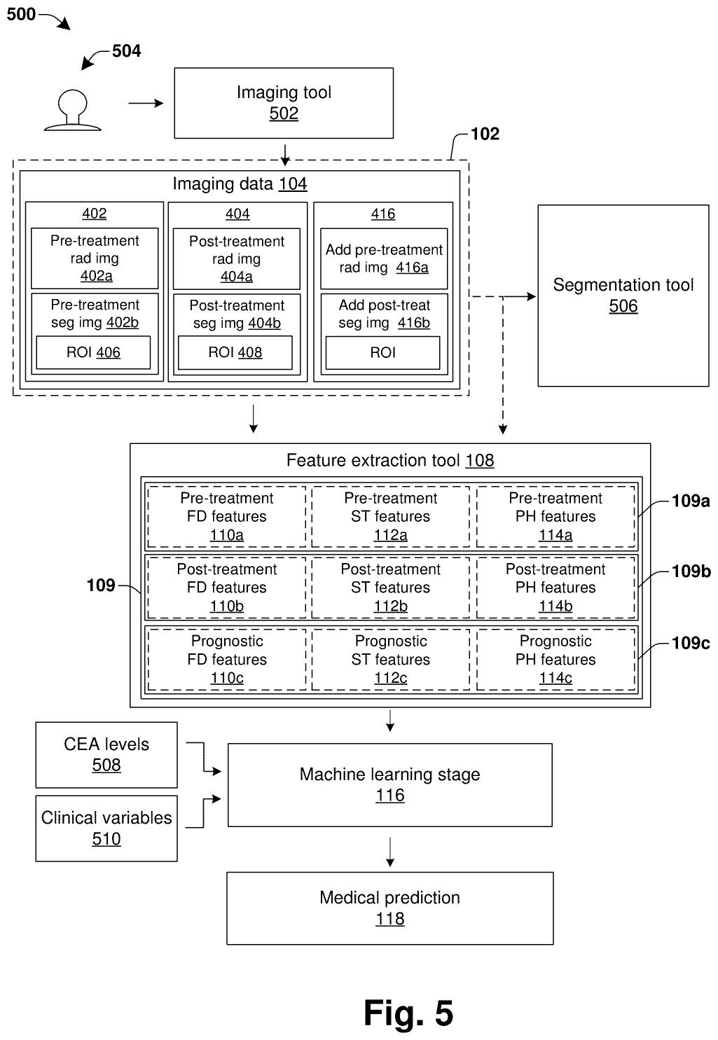

FIG. 5 illustrates some embodiments of a machine vision system 500 configured to utilize radiomic tumor diversity features to generate a medical prediction of a treatment response for a bowel cancer patient.

The machine vision system 500 comprises imaging data 104 from one or more bowel cancer patients 504 (e.g., rectal cancer patients). The imaging data 104 may comprise pre-treatment imaging data 402 and/or post-treatment imaging data 404. In various embodiments, the pre-treatment imaging data 402 and/or the post-treatment imaging data 404 may comprise a pre-treatment T2 (transverse relaxation time) weighted (T2w) MRI image. In some such embodiments, the T2w MRI image may be obtained using a T2-weighted turbo spin echo sequence.

In some embodiments, a segmentation tool 506 is configured to segment the imaging data 104 to identify one or more regions of interest (ROI). The segmentation tool 506 may segment one or more pre-treatment radiological images 402a to generate one or more one or more pre-treatment segmented images 402b that identify one or more first ROI 406 (e.g., respectively including a tumor region). The segmentation tool 506 may further segment one or more post-treatment radiological images 404a to generate one or more one or more post-treatment segmented images 404b that identify more second ROI 408 (e.g., including a rectal wall). In some embodiments, the segmented images may be stored in the imaging data 104 In some embodiments, the segmentation tool 506 may define a ROI for the pre-treatment imaging data 402 by identifying an entirety of a gross tumor volume (GTV) across all 2D sections on an axial or coronal imaging plane (through the long axis) of the tumor. In some embodiments, the segmentation tool 506 may define a ROI for the post-treatment imaging data 404 by annotating an entirety of a rectal wall for all 2D sections that best correspond with an original location of a tumor in a corresponding pre-treatment image.

A feature extraction tool 108 is configured to extract a plurality of tumor diversity features 109 from the imaging data 104. The plurality of tumor diversity features 109 may include pre-treatment features 109a extracted from the one or more first ROI 406 within the pre-treatment imaging data 402, post-treatment features 109b extracted from the one or more second ROI 408 within the post-treatment imaging data 404, and/or prognostic tumor diversity features 109c. The plurality of tumor diversity features 109 may include fractal dimension features 110a-110c, surface topology features 112a-112c, and persistent homology features 114a-114c. In some embodiments, the tumor diversity features 109 may comprise two-dimensional (2D) fractal features, three-dimensional (3D) topology features, and 2D persistence homology features. In other embodiments, the tumor diversity features 109 may comprise 3D fractal features, 3D topology features, and 3D persistence homology features.

A machine learning stage 116 is configured to operate upon an input vector including one or more of the plurality of tumor diversity features 109 (e.g., the pre-treatment features, or the post-treatment features 109b, or the prognostic tumor diversity features 109c) to generate a medical prediction 118 of a treatment response for the bowel cancer patient. In some embodiments, the machine learning stage 116 may comprise a deep learning model. In some embodiments, the machine learning stage 116 may comprise a regression model, a Cox Hazard regression model, a support vector machine, a linear discriminant analysis (LDA) classifier, a naïve Bayes classifier, or the like, run on one more processors. In some embodiments, the machine learning stage 116 may be run on one or more processors (e.g., a central processing unit including one or more transistor devices configured to operate computer code to achieve a result, a microcontroller, or the like).

In some embodiments, the input vector provided to the machine learning stage 116 may further comprise carcinoembryonic antigen levels (CEA levels). In such embodiments, the machine learning stage 116 may comprise one or more machine learning models configured to utilize one or more of the plurality of tumor diversity features 109 along with CEA levels 508 to generate the medical prediction 118. It has been appreciated that utilizing both tumor diversity features 109 and CEA levels 508 (e.g., using an input vector comprising a combined feature set including fractal dimension features, surface topology features, persistent homology features, and CEA features) provides for an improved reliability of the medical prediction 118. This may be because tumor diversity features 109 may reflect heterogeneity and self-similarity patterns of tumor cells and have been found to be associated with tumor prognosis whereas the CEA levels 508 characterize a biological behavior of a tumor, which may affect a tumor stage and/or prognosis.

In some embodiments, the input vector provided to the machine learning stage 116 may further comprise one or more clinical variables 510. In such embodiments, the machine learning stage 116 may further comprise one or more machine learning models configured to utilize one or more of the plurality of tumor diversity features 109 along with one or more clinical variables 510 such as membrane protein biomarkers, gene profiles, and tumor location, and/or the like in generating the medical prediction 118. The use of the one or more clinical variables 510 may help assess a potential for personalized treatment and their effects on the model performance.

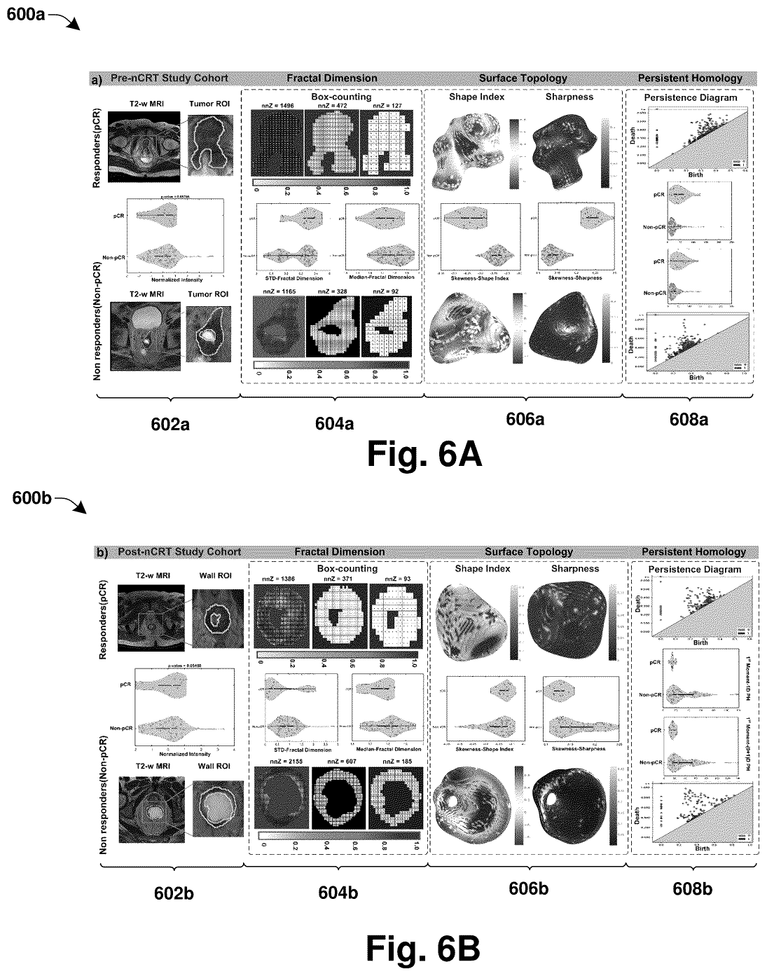

FIGS. 6A-6B illustrate some embodiments of flow charts showing tumor diversity features being extracted from pre-treatment images and post treatment images.

FIGS. 6A-6B depict representative visualizations of radiomic descriptor families 600a extracted from pre-treatment T2w MRI images 602a and of radiomic descriptor families 600b extracted from post-treatment T2w MRI images 602b. The radiomic descriptor families 600a-600b depict structural differences between rectal cancer patients experiencing a pathologic complete response (pCR) and rectal cancer patients experiencing a non-pathologic complete response (non-pCR) before and after long-course chemoradiation therapy.

The radiomic descriptor families include tumor diversity features relating to a fractal dimension 604a-604b, a surface topology 606a-606b, and a persistent homology 608a-608b. The middle row of FIGS. 6A-6B show normalized intensity and the statistical measures of top-ranked features from each descriptor family and their distribution (violin-plot) for pCR and non-pCR.

The fractal dimension 604a-604b quantifies tumor heterogeneity in terms of changes in structural details. In some embodiments, the fractal dimension may be calculated using a box-counting approach to compute an intrinsic dimensionality in an object for N number of points (e.g.,

$$FD = \frac{Log ( N )}{Log ( \frac{1}{r} )} ,$$

where r=size of a grid cell,

$$( \frac{1}{{2}^{j} for j = 1 , 2 … m} )$$

and N=points occupancy within the grid i, (Σᵢ₌₁^{max size of ROI}(point Count)ᵢ²)). For a given ROI, fractal dimension features may include statistics comprising mean, median, standard deviation, skewness, min, max, and entropy (e.g., across all boxes).

Surface topology 606a-606b investigates complex surface morphology of heterogeneous objects by constructing a mesh of vertices on the surface. Surface topology may include a curvedness and a sharpness. Curvedness describes an intensity of curvature (distance from origin to a planar point) and is inversely proportional to a size of the object. Sharpness relates to the inverse of a radius of curvature of an edge. Curvedness may include principal curvature (e.g., quantifying how a surface bends by different amounts in different directions at a given point in a plane), a Gaussian curvature, a mean curvature (e.g., describing a local shape geometry in terms of position and direction), and/or the like. For a given ROI, surface topology features may include statistics comprising mean, median, standard deviation, skewness, kurtosis computed (e.g., across all vertices in a mesh).

Persistent homology 608a-608b quantifies topological features that persist across multiple scales for shape description. Persistent homology may be computed using cubical complexes to characterize changes in relative structures of an object with respect to a parameter (e.g., a filtration unit). Features detected by persistent homology can be visualized by persistence pairs (Birth, Death∈({0, . . . m}∪∞) which represents non-null contribution in topology analysis. The value Death-Birth show ‘lifespan’ of the homology class (0D, 1D, 2D) and allows to identify the relevant classes for analysis. For a given ROI, persistent homology features may include raw moments of the d-b distribution (Moment 1, 2, 3, 4).

It has been appreciated that to generate a medical prediction for a rectal cancer treatment response, a disclosed machine vision system may utilize tumor diversity features including 2 fractal dimension features (e.g., standard deviation and median of fractal dimension), 2 surface topology features (e.g., skewness of shape index and sharpness), and 2 persistent homology features (e.g., 1st moment of 1D, and total (0D+1D) homology components). Such tumor diversity features are able to yield a high area under curve (AUC) that indicates a high degree of accuracy in a corresponding medical prediction.

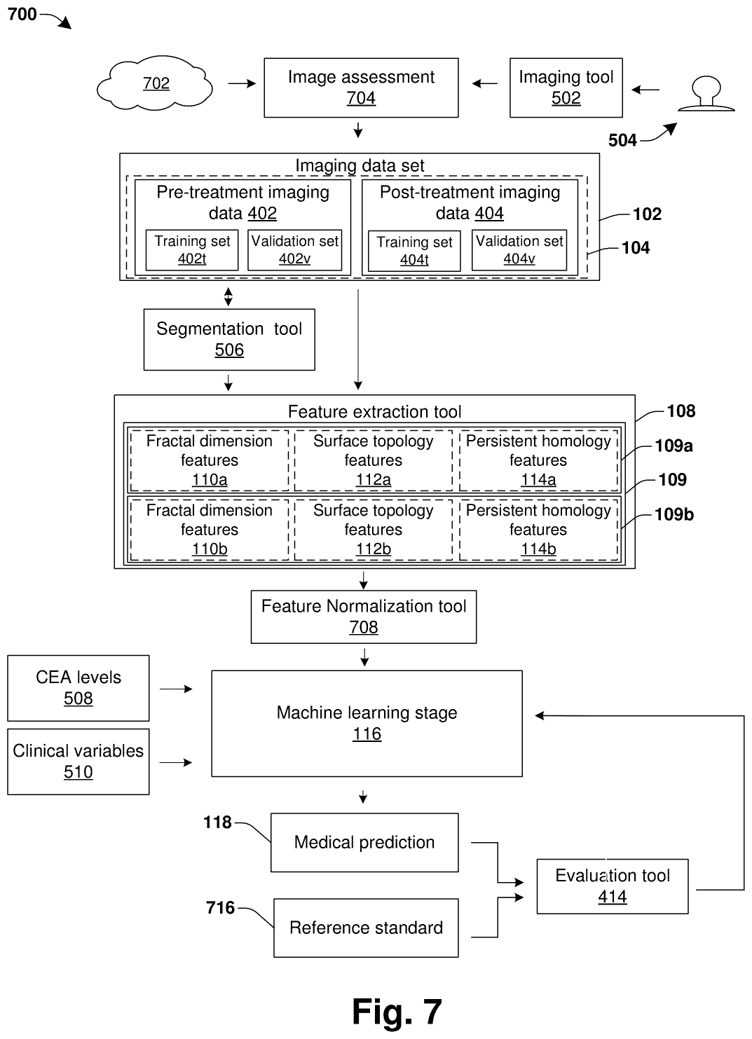

FIG. 7 illustrates some additional embodiments of a machine vision system 700 configured to utilize tumor diversity features to generate a medical prediction of a treatment response for a bowel cancer patient.

The machine vision system 700 comprises imaging data 104 from one or more bowel cancer patients 504. In various embodiments, the imaging data 104 may comprise pre-treatment imaging data 402 and post-treatment imaging data 404. The pre-treatment imaging data 402 may include a training data set 402t and a validation set 402v. The post-treatment imaging data 404 may include a training data set 404t and a validation set 404v. In various embodiments, the imaging data 104 may be obtained by an imaging tool 502 and/or from an on-line database 702 and/or archive containing radiological images from patients generated at different sites (e.g., different hospitals, research laboratories, and/or the like). In some embodiments, the imaging data 104 may be obtained from different models of scanners and/or from scanners manufactured by different scanner manufacturers. In some embodiments, the imaging data 104 may be obtained under varying imaging parameters (e.g., magnet strengths, repetition/echo times, in-plane resolutions, and slice thicknesses).

In some embodiments, prior to including radiological images within the imaging data 104, an image assessment tool 704 may be configured to apply one or more inclusion criteria and exclusion criteria to radiological images. In some embodiments, the image assessment tool 704 may be configured to minimize effects of varying voxel resolutions in different data sets by isotropically resampling the different data sets to a fixed voxel resolution (e.g., of 1×1×1 mm) to ensure a consistently sized ROI across all patients. Since the T2-weighted signal intensities are characteristically not quantitative and show significantly differing values for the reference regions between scanners and institutions, image intensities may be zero-mean normalized within a testing and validation set separately. In some embodiments, to standardize region of interest (ROI) definition, the image assessment tool 704 may utilize a slice comprising the largest cross-sectional tumor region for pre-neoadjuvant chemoradiation therapy (pre-nCRT) and the largest cross-sectional rectal wall region for post-neoadjuvant chemoradiation therapy (post-nCRT) as the imaging data 104.

A feature extraction tool 108 is configured to extract a plurality of tumor diversity features 109 from the imaging data 104. The plurality of tumor diversity features 109 include pre-treatment features 109a and post-treatment features 109b. In some embodiments, the tumor diversity features 109 may be extracted separately from pre-treatment imaging data 402 and the post-treatment imaging data 404 using MATLAB code run on one or more processors. In some embodiments, a total of 39 measurements capturing fractal dimensions, surface topology, and persistent homology may be computed within 2D ROIs from each of the one or more bowel cancer patients 504. In some embodiments, 7 statistical measures of fractal dimensions may be computed to capture self-similar patterns within a given lesion, 20 surface topology features representing object sharpness, shape index, curvature, and curvedness, and 12 persistent homology features corresponding to the number of active connected components with respect to varying dimensions and scales.

In some embodiments, a feature normalization tool 708 is configured to perform a zero-mean feature normalization on the tumor diversity features 109 to ensure that the tumor diversity features 109 are within a comparable range of values. The feature normalization tool 708 may separately perform the zero-mean feature normalization on the pre-treatment features 109a and the post-treatment features 109b. In some embodiments, the feature normalization tool 708 may perform the zero-mean feature normalization by subtracting a mean from the features sets and dividing by the mean absolute deviation.

A machine learning stage 116 is configured to operate upon the tumor diversity features 109 to generate a medical prediction 118 of a treatment response. In some embodiments, the machine learning stage 116 may comprise a pre-treatment machine learning model configured to generate a medical prediction 118 of a treatment response for a bowel cancer patient using one or more of the pre-treatment features 109a and a post-treatment machine learning model 412 configured to generate the medical prediction 118 using one or more of the post-treatment features 109b.

In some embodiments, the pre-treatment machine learning model and the post-treatment machine learning model are configured to identify a plurality of prognostic pre-treatment features and/or a plurality of prognostic post-treatment features that are highly (e.g., most) determinative of a pathologic complete response to treatment (e.g., neoadjuvant chemoradiation therapy). In some embodiments, the pre-treatment machine learning model and the post-treatment machine learning model may comprise a least absolute shrinkage and selection operator (LASSO) algorithm. In some embodiments, the extraction of the plurality of prognostic pre-treatment features and/or the plurality of prognostic post-treatment features and the medical prediction of treatment response for a bowel cancer patient may be coincidentally performed using a Cox Hazard regression model with least absolute shrinkage and selection operator (LASSO) feature select.

In some embodiments, for each patient pathologists may assess and record tumor-node metastasis staging (e.g., using ypTNM classification) and/or tumor regression grade of excised specimens (e.g., according to the AJCC staging manual) into a clinical report. This assessment of postsurgical specimens may be used as the reference standard 716 for tumor response to nCRT. An evaluation tool 414 may compare the reference standard 716 to the medical prediction 118 of the treatment response to identify the prognostic pre-treatment features and/or the prognostic post-treatment features.

In some embodiments, prognostic tumor diversity features may be selected based upon the prognostic pre-treatment features and/or the prognostic post-treatment features. In some embodiments, the prognostic pre-treatment features and/or the prognostic post-treatment features may be analyzed to identify the prognostic tumor diversity features that reflect response-related evolution differences between pre-treatment images and post-treatment images. In some embodiments, for each tumor diversity feature a Wilcoxon rank sum statistical testing may be used to identify significant differences between values of pre-treatment and post-treatment feature values. This may be done separately for pCR and non-pCR patients to identify the prognostic tumor diversity features.

In some embodiments, the prognostic tumor diversity features may be a standard deviation and a median of fractal dimensions, the surface topology features comprise a skewness of object sharpness and a skewness of object shape index, and the persistent homology features comprise moment 1 of 1D and (0+1) D topological features. It has been appreciated that a combined feature set including fractal dimension features, surface topology features, and persistent homology features is able to achieve better results than any feature sets consisting solely of fractal dimension features, surface topology features, or persistent homology features.

The tumor diversity features 109 corresponding to the prognostic tumor diversity features may be subsequently extracted from the training and testing data and used to train a machine learning model (e.g., a combined machine learning model) for use on additional patients, since identified prognostic tumor diversity features have an ability to reflect response-related evolution differences between pre-treatment images and post-treatment images. In some embodiments, the prognostic tumor diversity features may be provided to the feature extraction tool 108, so that the feature extraction tool 108 may subsequently extract the tumor diversity features 109 to be the same as prognostic tumor diversity features.

The feature extract tool 108 may subsequently extract the tumor diversity features 109 from additional images (e.g., additional pre-treatment and post-treatment images obtained from an additional bowel cancer patient during routine examinations). The tumor diversity features 109 are then provided to the machine learning stage 116, which is configured to operate upon the identified tumor diversity features 109 to generate a medical prediction of a treatment response for the additional bowel cancer patient.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

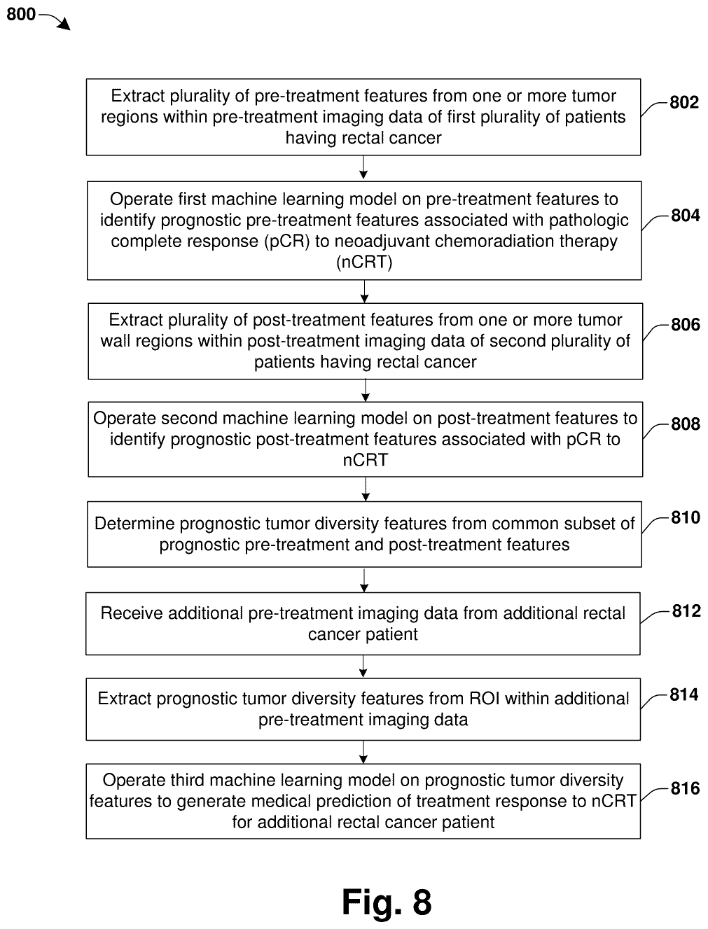

FIG. 8 illustrates some embodiments of a flow diagram showing a method 800 for using tumor diversity features to generate a medical prediction of a treatment response for a patient having rectal cancer.

At act 802, a plurality of pre-treatment features are extracted from one or more tumor regions within pre-treatment imaging data of a first plurality of patients having rectal cancer.

At act 804, a first machine learning model is operated upon the plurality of pre-treatment features to identify prognostic pre-treatment features associated with a pathologic complete response to neoadjuvant chemoradiation therapy for rectal cancer.

At act 806, a plurality of post-treatment features are extracted from one or more tumor wall regions within post-treatment imaging data of a second plurality of patients having rectal cancer.

At act 808, a second machine learning model is operated upon the plurality of post-treatment features to identify prognostic post-treatment features associated with a pathologic complete response to neoadjuvant chemoradiation therapy for rectal cancer.

At act 810, prognostic tumor diversity features are determined to include a common subset of the prognostic pre-treatment features and the prognostic post-treatment features.

At act 812, additional pre-treatment imaging data is received from an additional rectal cancer patient.

At act 814, the prognostic tumor diversity features are extracted from one or more regions of interest within the additional pre-treatment imaging data.

At act 816, a third machine learning model is operated on the prognostic tumor diversity features to generate a medical prediction of treatment response for the additional rectal cancer patient.

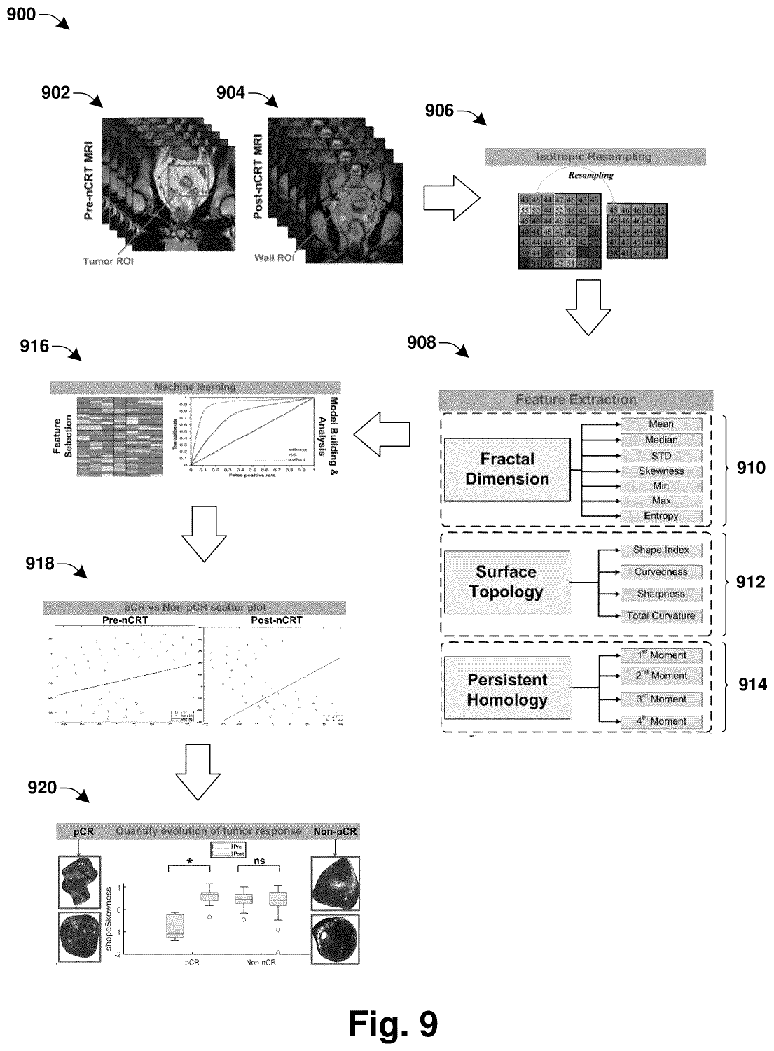

FIG. 9 illustrates some embodiments of a radiomic analysis pipeline 900 within a disclosed machine vision system configured to utilize tumor diversity features to generate a medical prediction of a treatment response for a rectal cancer patient.

The radiomic analysis pipeline 900 comprises formation of a first cohort 902 including MRI images taken before neoadjuvant chemoradiation therapy has been received (e.g., pre-nCRT images) and a second cohort 904 including MRI images taken after neoadjuvant chemoradiation therapy has been received (e.g., post-nCRT images). The first cohort 902 comprises one or more ROI including a tumor region. The second cohort 904 comprises one or more ROI including a rectal wall region.

The radiomic analysis pipeline 900 further comprises pre-processing 906 to account for resolution differences between images. In some embodiments, the pre-processing 906 may include isotropic resampling to minimize the effect of varying voxel resolutions in the cohorts. For example, patient datasets and annotations may be isotropically resampled to a fixed voxel resolution of 1×1×1 mm to ensure a consistently sized ROI across all patients.

The radiomic analysis pipeline 900 further comprises feature extraction 908. The feature extraction 908 may include extracting a plurality of pre-treatment and post-treatment features by determining statistical measures of fractal dimensions 910, surface topology 912, and persistent homology 914 measurements within the resampled images.

The radiomic analysis pipeline 900 further comprises machine learning 916. The machine learning 916 is configured to select prognostic pre-treatment and prognostic post-treatment features from the plurality of pre-treatment and post-treatment features, respectively. Machine learning models are trained on the prognostic pre-treatment and prognostic post-treatment features to provide an accurate medical prediction relating to a treatment response.

Based upon the machine learning models, the radiomic analysis pipeline 900 separately identifies tumor diversity features associated with pCR from each of the prognostic pre-treatment and prognostic post-treatment features (918). In both analyses, a definition of pCR may be identical (ypT0-1N0M0 or ypTRG0). To identify the tumor diversity features, machine learning analysis may involve initial Pearson correlation testing to reduce intra-class feature redundancy within each feature family (i.e., FD, ST, and PH), and Wilcoxon-based feature selection to identify tumor diversity features most frequently associated with pCR within each discovery cohort (separately for pre- and post-nCRT cohorts), across 50 runs of 3-fold cross-validation. Radiomic features may be rank ordered based on a frequency of selection across all cross-validation runs to select the most consistently identified descriptors from each feature family while being resilient to cohort variations. Separate feature sets may be constructed for the pre-nCRT and post-nCRT settings, respectively, while varying the number of features selected per feature family.

The radiomic analysis pipeline 900 evaluates top-ranked features for capturing differences in tumor response between pCR and non-pCR patients, both before and after neoadjuvant chemoradiation therapy (920).

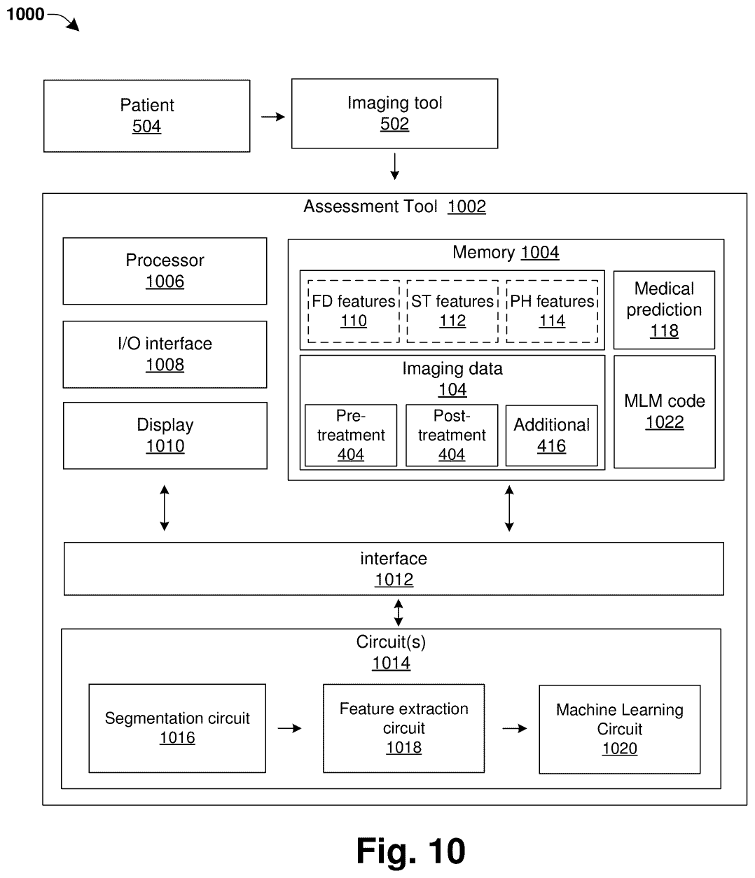

FIG. 10 illustrates a block diagram of some embodiments of a prognostic apparatus 1000 configured to utilize tumor diversity features to generate a medical prediction of a treatment response for a bowel cancer patient.

The prognostic apparatus 1000 comprises an assessment tool 1002. In some embodiments, the assessment tool 1002 is coupled to an imaging tool 502 (e.g., an MRI scanner, a CT scanner, and/or the like) that is configured to generate one or more radiological images corresponding to one or more bowel cancer patients 504.

The prognostic apparatus 1000 comprises a processor 1006 and a memory 1004. The processor 1006 can, in various embodiments, comprise circuitry such as, but not limited to, one or more single-core or multi-core processors. The processor 1006 can include any combination of general-purpose processors and dedicated processors (e.g., graphics processors, application processors, etc.). The processor 1006 can be coupled with and/or can comprise memory (e.g., memory 1004) or storage and can be configured to execute instructions stored in the memory 1004 or storage to enable various apparatus, applications, or operating systems to perform operations and/or methods discussed herein.

The memory 1004 may be configured to store imaging data 104 comprising pre-treatment imaging data and post-treatment imaging data. The imaging data 104 may include a plurality of imaging units (e.g., pixels, voxels, etc.) respectively having an associated intensity pixels. In some additional embodiments, the imaging data 104 may be stored in the memory 1004 as one or more training sets, testing sets, and/or validation sets of radiological images for training a machine learning circuit.

The prognostic apparatus 1000 also comprises an input/output (I/O) interface 1008 (e.g., associated with one or more I/O devices), a display 1010, one or more circuits 1014, and an interface 1012 that connects the processor 1006, the memory 1004, the I/O interface 1008, the display 1010, and the one or more circuits 1014. The I/O interface 1008 can be configured to transfer data between the memory 1004, the processor 1006, the one or more circuits 1014, and external devices (e.g., imaging tool 502).

In some embodiments, the one or more circuits 1014 may comprise hardware components. In other embodiments, the one or more circuits 1014 may comprise software components. In such embodiments, the one or more circuits 1014 may execute code (e.g., machine learning code 1022) stored in the memory 1004. The one or more circuits 1014 may comprise a segmentation circuit 1016. The segmentation circuit 1016 is configured to operate upon imaging data 104 to identify one or more regions of interest. For example, the segmentation circuit 1016 may identify one or more first ROI within the pre-treatment imaging data 402 and one or more second ROI within the post-treatment imaging data 404.

In some additional embodiments, the one or more circuits 1014 may further comprise a feature extraction circuit 1018. In some embodiments, the feature extraction circuit 1018 is configured to extract a plurality of tumor diversity features from the one or more ROI within the imaging data 104. The plurality of tumor diversity features may include fractal dimension features 110, surface topology features 112, and persistent homology features 114. In some additional embodiments, the one or more circuits 1014 may further comprise a machine learning circuit 1020. In some embodiments, the machine learning circuit 1020 is configured to utilize the plurality of tumor diversity features to generate a medical prediction 118 of a treatment response.

Therefore, the present disclosure relates an apparatus and/or method that utilizes a plurality of tumor diversity features, which have been extracted from one or more radiological images (e.g., MRI images) of a bowel cancer patient, to generate a medical prediction of the patient's response to treatment (e.g., neoadjuvant chemoradiation therapy).

In some embodiments, the present disclosure relates to a method. The method includes extracting a plurality of pre-treatment features from one or more first regions of interest (ROI) within pre-treatment imaging data; identifying prognostic pre-treatment features from the plurality of pre-treatment features, the prognostic pre-treatment features being determinative of a treatment response; extracting a plurality of post-treatment features from one or more second ROI within post-treatment imaging data; identifying prognostic post-treatment features from the plurality of post-treatment features, the prognostic post-treatment features being determinative of the treatment response; determining prognostic tumor diversity features from a common subset of the prognostic pre-treatment features and the prognostic post-treatment features; and operating a machine learning stage to generate a medical prediction of the treatment response for a bowel cancer patient using the prognostic tumor diversity features.

In other embodiments, the present disclosure relates to a non-transitory computer-readable medium storing computer-executable instructions that, when executed, cause a processor to perform operations, including extracting a plurality of tumor diversity features from imaging data from a patient having rectal cancer, the plurality of tumor diversity features including radiomic features that describe changes in structural related patterns within a tumor due to a treatment response; and operating a machine learning stage on an input vector including the plurality of tumor diversity features to generate a medical prediction of the treatment response for the patient, the medical prediction of the treatment response including a prediction of either a pathologic complete response or a non-pathologic complete response.

In yet other embodiments, the present disclosure relates to an apparatus including a memory configured to store pre-treatment imaging data and post-treatment imaging data of bowel cancer patients; a feature extraction tool configured to extract a plurality of pre-treatment features from the pre-treatment imaging data and to further extract a plurality of post-treatment features from the post-treatment imaging data; a machine learning stage configured to identify prognostic pre-treatment features from the plurality of pre-treatment features and to further identify prognostic post-treatment features from the plurality of post-treatment features, the prognostic pre-treatment features and the prognostic post-treatment features being determinative of a treatment response; and an evaluation tool configured to determine prognostic tumor diversity features from the prognostic pre-treatment features and the prognostic post-treatment features.

Embodiments discussed herein relate to training and/or employing machine learning models (e.g., unsupervised (e.g., clustering) or supervised (e.g., classifiers, etc.) models) to determine a medical prediction based on a combination of radiomic features and deep learning, based at least in part on features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
extracting a plurality of pre-treatment features from one or more first regions of interest (ROI) within pre-treatment imaging data;
identifying prognostic pre-treatment features from the plurality of pre-treatment features, the prognostic pre-treatment features being determinative of a treatment response;
extracting a plurality of post-treatment features from one or more second ROI within post-treatment imaging data;
identifying prognostic post-treatment features from the plurality of post-treatment features, the prognostic post-treatment features being determinative of the treatment response;
determining prognostic tumor diversity features from a common subset of the prognostic pre-treatment features and the prognostic post-treatment features; and
operating a machine learning stage to generate a medical prediction of the treatment response for a bowel cancer patient using the prognostic tumor diversity features.

2. The method of claim 1, wherein the medical prediction of the treatment response is a prediction of either a pathologic complete response or a non-pathologic complete response.

3. The method of claim 1, wherein the prognostic tumor diversity features have differences in values between the pre-treatment imaging data and the post-treatment imaging data that exceed a threshold.

4. The method of claim 1, wherein the one or more first ROI include a tumor region of the pre-treatment imaging data.

5. The method of claim 1, wherein the one or more second ROI include a rectal wall of the post-treatment imaging data.

6. The method of claim 1,
wherein the pre-treatment imaging data is obtained prior to applying neoadjuvant chemoradiation therapy for locally advanced rectal cancer; and
wherein the post-treatment imaging data is obtained after applying the neoadjuvant chemoradiation therapy for the locally advanced rectal cancer.

7. The method of claim 1, further comprising:
performing a zero-mean feature normalization to the prognostic tumor diversity features.

8. The method of claim 1, wherein the prognostic tumor diversity features comprise statistical measures of fractal dimensions and surface topology.

9. The method of claim 1, further comprising:
operating the machine learning stage to generate the medical prediction of the treatment response using both the prognostic tumor diversity features and carcinoembryonic antigen (CEA) levels.

10. The method of claim 1, wherein the prognostic tumor diversity features include fractal dimension features, surface topology features, and persistent homology features.

11. The method of claim 10, wherein the fractal dimension features comprise a standard deviation and a median of fractal dimensions, the surface topology features comprise a skewness of object sharpness and a skewness of object shape index, and the persistent homology features comprise moment 1 of 1D and (0+1) D topological features.

12. A non-transitory computer-readable medium storing computer-executable instructions that, when executed, cause a processor to perform operations, comprising:
extracting a plurality of tumor diversity features from imaging data from a patient having rectal cancer, the plurality of tumor diversity features including radiomic features that describe changes in structural related patterns within a tumor due to a treatment response; and
operating a machine learning stage on an input vector including the plurality of tumor diversity features to generate a medical prediction of the treatment response for the patient, wherein the medical prediction of the treatment response includes a prediction of either a pathologic complete response or a non-pathologic complete response.

13. The non-transitory computer-readable medium of claim 12, wherein the input vector for the machine learning stage further comprises carcinoembryonic antigen levels.

14. The non-transitory computer-readable medium of claim 12, wherein the input vector for the machine learning stage further comprises one or more clinical variables.

15. The non-transitory computer-readable medium of claim 12, wherein the operations further comprise:
extracting a plurality of pre-treatment features from one or more first regions of interest (ROI) within pre-treatment imaging data;
identifying prognostic pre-treatment features, which are determinative of the treatment response, from the plurality of pre-treatment features;
extracting a plurality of post-treatment features from one or more second ROI within post-treatment imaging data;
identifying prognostic post-treatment features, which are determinative of the treatment response, from plurality of post-treatment features; and
determining prognostic tumor diversity features from a common subset of the prognostic pre-treatment features and the prognostic post-treatment features, wherein the plurality of tumor diversity features include the prognostic tumor diversity features.

16. The non-transitory computer-readable medium of claim 15, wherein the prognostic tumor diversity features have differences in values between the pre-treatment imaging data and the post-treatment imaging data that exceed a threshold.

17. An apparatus, comprising:
a memory configured to store pre-treatment imaging data and post-treatment imaging data of bowel cancer patients;
a feature extraction tool configured to extract a plurality of pre-treatment features from the pre-treatment imaging data and to further extract a plurality of post-treatment features from the post-treatment imaging data;
a machine learning stage configured to identify prognostic pre-treatment features from the plurality of pre-treatment features and to further identify prognostic post-treatment features from the plurality of post-treatment features, the prognostic pre-treatment features and the prognostic post-treatment features being determinative of a treatment response; and
an evaluation tool configured to determine prognostic tumor diversity features from the prognostic pre-treatment features and the prognostic post-treatment features.

18. The apparatus of claim 17, wherein the machine learning stage is further configured to operate upon the prognostic tumor diversity features to generate a medical prediction corresponding to the treatment response.

19. The apparatus of claim 18, wherein the machine learning stage is further configured to operate upon carcinoembryonic antigen (CEA) levels to generate the medical prediction of the treatment response.

20. The apparatus of claim 17, wherein the prognostic tumor diversity features comprise a standard deviation and a median of fractal dimensions, a skewness of object sharpness and a skewness of object shape index, and moment 1 of 1D and (0+1) D topological features.
